(19) United States
(12) Patent Application Publication
(10) Pub. No.: US 2023/0186098 A1
(43) Pub. Date: Jun. 15, 2023

(54) DISTRIBUTED GENERATIVE ADVERSARIAL NETWORKS SUITABLE FOR PRIVACY-RESTRICTED DATA

(71) Applicant: Rutgers, The State University of New Jersey, New Brunswick, NJ (US)

(72) Inventors: Qi CHANG, Princeton, NJ (US); Dimitris METAXAS, Princeton, NJ (US); Hui QU, Piscataway, NJ (US); Yikai ZHANG, Edison, NJ (US)

(21) Appl. No.: 17/926,457

(22) PCT Filed: May 27, 2021
(86) PCT No.: PCT/US21/34414

§ 371 Date: Nov. 18, 2022

Related U.S. Application Data
(60) Provisional application No. 63/030,467, filed on May 27, 2020.

(51) Int. Cl. G06N 3/091; G16H 30/40

(52) U.S. Cl. CPC G06N 3/091; G16H 30/40

(57) ABSTRACT

An asynchronous distributed generative adversarial network (AsynDGAN) can include a central computing system and at least two discriminator nodes. The central computing system can include a generator neural network, an aggregator, and a network interface. Each discriminator node can have its own corresponding training data set. In addition, different discriminator nodes can use different data modalities. The central computing system communicates with each of the at least two discriminator nodes via the network interface and aggregates data received from the at least two discriminator nodes, via the aggregator, to update a model for the generator neural network during training of the generator neural network. The central computing system can further include a data access system that supports third party access to synthetic data generated by the generator neural network.

### CROSS REFERENCE TO RELATED APPLICATION

This application claims the benefit of U.S. Provisional Patent Application No. 63/030,467, filed May 27, 2020.

### BACKGROUND

Privacy-restricted data such as medical records create a challenge for training machine learning algorithms. For example, in the United States, medical images are subject to HIPAA (Health Insurance Portability and Accountability Act) and other privacy rules, including those put in place by the Institutional Review Board (IRB) of the Food and Drug Administration, that restrict who has access to the images and how the images are stored. Similarly, in the European Union and the European Economic Area, GDPR (General Data Protection Regulation) places restrictions on personal data (personal identifiable information PII) such as those found in medical images. The restrictions in place by the United States (at the Federal and State level) and many other countries that do not permit patient data to leave their country further limit the creation of large data sets of medical images and sharing between institutions.

Sufficient data volume is necessary for training a successful machine learning algorithm for medical image analysis. Currently, the available data sets for machine learning research are still very limited: the largest set of medical image data available to public is 32 thousand CT images, which is only 0.02% of the annual acquired images in the United States. In contrast, the ImageNet project (Senior Research Team of Li Fei-Fei, Jia Deng, Olga Russakovsky, Alex Berg, and Kai Li), which is a large visual data set designed for use in visual object recognition research, has more than 14 million images that have been annotated in more than 20,000 categories. Therefore, for medical related applications, there can be a benefit to generating synthetic data that can be used to train machine learning algorithms. However, training the machine learning algorithms to generate decent synthetic data for medical applications is a challenge.

### BRIEF SUMMARY

A data privacy-preserving and communication efficient distributed generative adversarial network (GAN) is described.

The distributed GAN can be referred to as an asynchronous distributed GAN (AsynDGAN) and can include a central computing system and at least two discriminator nodes. The central computing system can include a generator neural network, an aggregator, and a network interface. Each discriminator node can have its own corresponding training data set. The central computing system communicates with each of the at least two discriminator nodes via the network interface and aggregates data received from the at least two discriminator nodes, via the aggregator, to update a model for the generator neural network during training of the generator neural network. The central computing system can further include a data access system that supports third party access to synthetic data generated by the generator neural network. Application programming interfaces (APIs) can be provided as part of the data access system.

The generator neural network of the AsynDGAN can be trained by generating, at a generator neural network, a fake image using a model; sending the fake image to at least one discriminator node of a plurality of discriminator nodes, each discriminator node having its own training data set; receiving at least one gradient generated with respect to the fake image, each gradient being from a corresponding discriminator node of the at least one discriminator node of the plurality discriminator nodes; updating the model at the generator neural network using the received at least one gradient; and iterating the generating of an updated fake image, sending the updated fake image to the at least one discriminator node, and the updating of the model using a new gradient received from the at least one discriminator node until an iteration condition is satisfied. Multi-modality fake images are able to be trained and generated at the generator by using a modality bank of parameters associated with each modality. Discriminator nodes can use the fake images of their corresponding modality preferences and the gradients generated at the discriminator node can be used to train across all modalities at the generator.

This Summary is provided to introduce a selection of concepts in a simplified form that are further described below in the Detailed Description. This Summary is not intended to identify key features or essential features of the claimed subject matter, nor is it intended to be used to limit the scope of the claimed subject matter.

### BRIEF DESCRIPTION OF THE DRAWINGS

FIG. 1A illustrates a basic GAN configuration.

FIG. 1B illustrates a simplified distributed GAN configuration.

FIG. 2 illustrates a central computing system for an AsynDGAN.

FIG. 3 illustrates a training method for a centralized generator of the AsynDGAN.

FIGS. 4A and 4B illustrate an overview of an AsynDGAN architecture applied to privacy-restricted image applications.

FIG. 5 shows an example training algorithm for a distributed GAN.

FIGS. 6A-6D illustrate an optimization process for a distributed GAN.

FIG. 7 illustrates an AsynDGAN architecture incorporating a modality bank.

FIGS. 8A-8C show plots of the learned distributions in accordance with an experiment on synthetic data.

FIG. 9 shows images from the quantitative brain tumor segmentation results with respect to each of the four methods of the experiment.

FIG. 10 shows examples of synthetic images from AsynDGAN as part of the quantitative brain tumor segmentation experiment.

FIG. 11 shows images from the nuclei segmentation results with respect to each of the four methods of the experiment.

FIG. 12 shows examples of synthetic nuclei images from AsynDGAN.

FIG. 13 shows examples of synthetic images from AsynDGAN with modality bank as part of a multi-modality brain tumor segmentation experiment.

FIG. 14 shows examples of synthetic images from AsynDGAN with modality bank as part of a multi-modality brain tumor segmentation experiment using missing modality data sets.

### DETAILED DESCRIPTION

A data privacy-preserving and communication efficient distributed GAN is described. This distributed GAN framework uses a centralized generator and distributed discriminators to learn the generative distribution of a target data set. Synthetic images that are generated by the centralized generator can then be used for other machine learning algorithms. The described framework can be referred to as a distributed asynchronous discriminator GAN (AsynDGAN).

Learning from synthetic images has several advantages including a privacy mechanism, unrestricted (by privacy laws) data sharing, and adaptivity to architecture updates.

Regarding the privacy mechanism, the centralized generator has limited information for the raw images at the locations of each discriminator. When the generator communicates with discriminators at each location, information about the synthetic image is transmitted, not the raw data. Such a mechanism prohibits the centralized generator's direct access to raw data; thus, securing privacy.

A challenge with certain existing approaches to machine learning with data privacy (e.g., Federated Learning using random noise for privacy) is the constraints a network may place on communications involving model information (e.g., parameters and gradients—having a dimension d that may involve millions of parameters). In addition, some methods (e.g., a split learning approach that separates shallow and deep layers and uses a data block for privacy) do not apply to neural networks with skip connections.

In the AsynDGAN framework described herein, the communication cost in each iteration is free of the dimension d. Only auxiliary data (label/mask and gradient), ‘fake’ data and discriminator losses are passed between the centralized processor and local nodes in the network. For example, for a 128×128 size gray-scale image, communication cost per iteration for each node is 8 mb with batch size 128. Since the centralized generator has only access to discriminator and auxiliary data, the privacy of client is secured via a separating block mechanism.

Regarding the sharing of synthetic images without restriction, it is possible to aggregate and redistribute the synthetic data at the central system, which—for medical data—can result in a publicly accessible and faithful medical database. The inexhaustible database can benefit researchers and practitioners, as well as boost the development of medical intelligence. Indeed, appropriate synthetic images can be used by any type of application that requires training where privacy concerns are an issue such as in the financial and healthcare industries (as well as certain military industries) or where an enormous data set is required to train machine learning algorithms. Synthetic data generated using the described framework may be used to train AI algorithms to identify medical conditions, to train fraudulent activity detection systems to protect financial activities, to test software and ensure quality, and to aid self-driving technology, as some examples.

Regarding adaptivity to architecture updates, machine learning architectures evolve rapidly to achieve better performance. For example, improvements can be made using loss functions, network modules, and optimizers. As the machine learning architectures change, it can be a challenge to train the new model since the private-sensitive data may not be always accessible. So even if it was possible to originally train a model based on these data sets, it may not be possible to embrace new architectures to achieve higher performance. Instead of training a task-specific model, the described method trains a generator that learns from distributed discriminators. Specifically, the distribution of private data sets is learned by a generator to produce synthetic images for future use, without worrying about the loss of the proprietary data sets. Moreover, the described central generator can be used for multi-modality data while leveraging the distributed data sets.

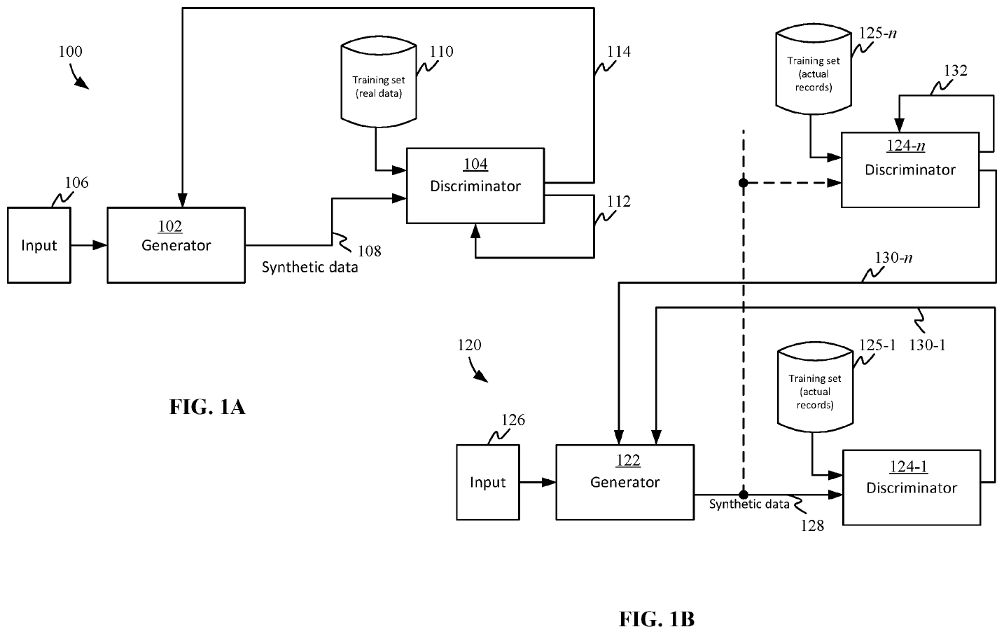

FIG. 1A illustrates a basic GAN configuration. A GAN 100 includes a generator neural network (“generator”) 102 and a discriminator neural network (“discriminator”) 104. The generator 102 takes input 106 and generates new data instances and the discriminator 104 evaluates the new data instances for authenticity (e.g., identifying fake data from real data). Through backpropagation, the discriminator's classification can provide a signal that the generator 102 uses to update its weights. In other words, a GAN estimates generative distribution via the discriminator (also referred to as an adversarial supervisor). Specifically, the generator 102 attempts to imitate the data from target distribution to make the ‘fake’ data indistinguishable to the discriminator 104.

The discriminator 104 is trained using real data 110 as positive examples and synthetic data 108 as negative examples. The synthetic data 108 used for the training is generated by the generator 102 while maintaining constant weights. During training, the discriminator 104 classifies the data (both the real data 110 and the synthetic data 108) and uses backpropagation of only the discriminator loss 112 to update its model (e.g., the weights for the discriminator network). The discriminator loss 112 penalizes the discriminator for misclassifying the data.

The generator 102 is trained using feedback from the discriminator 104. An initial input (usually a form of noise) is first used to create synthetic data (based on some initial weights at the generator neural network) and this synthetic data is sent to the discriminator 104 to classify. The discriminator 104 produces a generator loss 114 (which was ignored during the discriminator training) that is backpropagated through the discriminator 104 and generator 102 to obtain gradients. The gradients are then used to adjust the weights for the generator neural network.

FIG. 1B illustrates a simplified distributed GAN configuration. Referring to FIG. 1B, a distributed GAN 120 includes a centralized generator neural network (“centralized generator”) 122 that is supervised by multiple discriminator neural networks (“discriminator nodes”) 124-1, . . . , 124-n. Each discriminator node 124-1, . . . , 124-n can access its own set of real data (e.g., in corresponding training sets of real data 125-1, . . . , 125-n) and may be managed by a different entity. For example, each discriminator node 124-1, . . . , 124-n can be associated with a corresponding medical center with access to its own, local data.

The centralized generator 122 takes input 126 and generates synthetic data 128 that is evaluated by each discriminator node 124-1, . . . , 124-n. The discriminators evaluate the synthetic data 128 received from the generator 122 and return a gradient 130-1, . . . , 130-n, which is used to adjust the weights of the generator neural network 122. The discriminators can each be trained on their own training sets of real data 125-1, . . . , 125-n, for example, by backpropagating a gradient-based update 132.

Accordingly, synthetic records may be generated by the generator 122 and then evaluated by each discriminator node 124-1, . . . , 124-n to identify how close the synthetic records are to actual records. During training of the generator, a goal is to generate a synthetic record that at least one discriminator indicates has a high probability of being authentic. In some cases, the generator requires all discriminators to indicate a probability above a certain threshold. In some cases, the generator requires a subset of the discriminators to indicate the probability above the certain threshold. In some cases, it is sufficient for a single discriminator to indicate the high probability (e.g., above a certain threshold) of being authentic. The resulting synthetic records can be used to increase the data sets used for other algorithms and applications.

In the distributed GAN configuration, only the synthetic data, losses, masks (e.g., in the communication with the synthetic data to the discriminators), and gradients are needed to be transferred between the centralized generator 122 and the remote entities with their corresponding discriminator nodes 124-1, . . . , 124-n. This means that less data is required to be transmitted, and the communications are more secure/ensure privacy than if the communications involved the entire data (e.g., entire image). Of course, some implementations can include additional data that is transmitted. In addition, some implementations can involve adding additional security to the communications (e.g., techniques such as differential privacy and homomorphic encryption can be applied to secure the exchange data).

Because medical records are privacy restricted, it is difficult to have a large enough data set of actual records from which a generator can be trained. That is, even if one institution was able to train a generator locally on a set of medical records that the institution manages, there is likely insufficient numbers of records to properly train the generator to obtain high quality enough synthetic records.

Advantageously, in the health entities learning context, the described framework can aggregate data sets from multiple hospitals to obtain a faithful estimation of the overall distribution. A specific task (e.g., segmentation and classification) can be accomplished locally by acquiring data from the generator.

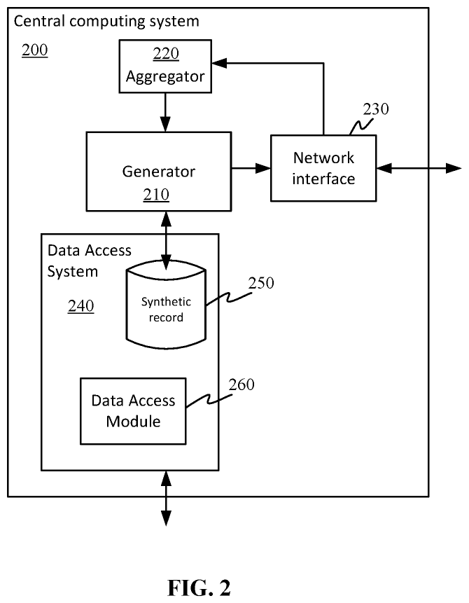

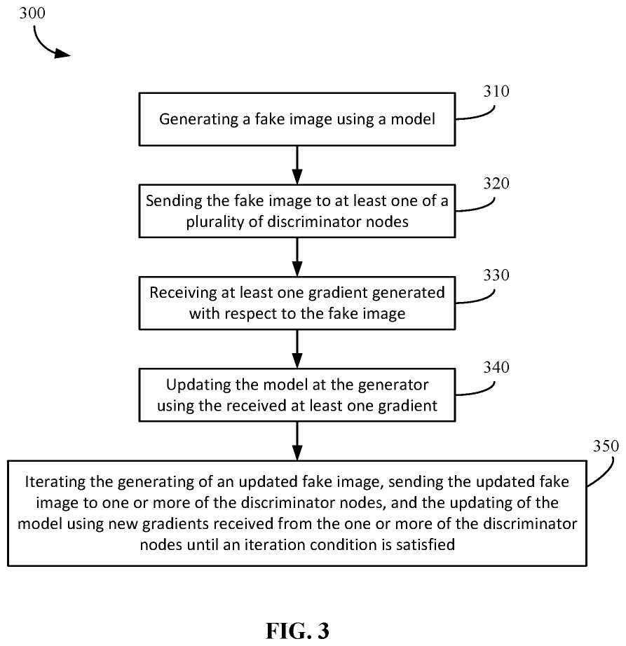

FIG. 2 illustrates a central computing system for an AsynDGAN; and FIG. 3 illustrates a training method for a centralized generator of the AsynDGAN. Referring to FIG. 2, a central computing system 200 for an AsynDGAN can include a generator neural network 210, an aggregator 220, and a network interface 230. The computing system 200 communicates with each of at least two discriminator nodes via the network interface 230. In some cases, the central system can receive, via the network interface 230 a training data request from a discriminator node of the at least two discriminator nodes. The training data request can be a request for the generator neural network 210 to generate a training synthetic data and transmit the training synthetic data for that particular discriminator. In some cases, the central computing system 200 does a push request to the discriminators to indicate that the discriminator is receiving training data and should perform a training process. New discriminators may be onboarded through a registration process. Registration can include information on the type of data to be used. For example, in a medical context, the registration can include information such as imaging modality/modalities, machine parameters (e.g., related to the imaging machines), window, organ(s) of interest, and disease.

The computing system 200 aggregates data received from the at least two discriminator nodes, via the aggregator 220, to update a model (e.g., weights) for the generator neural network 210 during training of the generator neural network. The model is stored in storage media at the computing system 200 and can include parameters specific for the type of data (and features of interest). The generator neural network 210 can be a common domain agnostic network where multiple models can be stored, updated using the same data received from the discriminator nodes, and used to support multiple modalities. That is, the computing system 200 can include a modality bank storing style parameters for a plurality of models, including the model for the generator neural network 210. The style parameters for the plurality of models can, for example, correspond to at least two respective image modalities. Each of the plurality of models can be updated using a same data received from the at least two discriminator nodes.

For example, as illustrated in FIG. 3, a training method 300 can include generating (310) a fake image using a model; sending (320) the fake image to at least one of a plurality of discriminator nodes, each discriminator node of the plurality of discriminator nodes having its own training data set; receiving (330) at least one gradient generated by the at least one of the plurality of discriminator nodes with respect to the fake image, each gradient being from a corresponding discriminator node of the at least one of the plurality of discriminator nodes; updating (340) the model at the generator neural network using the received at least one gradient; and iterating (350) the generating of an updated fake image, sending the updated fake image to the at least one discriminator node, and the updating of the model using new gradients received from the at least one discriminator node until an iteration condition is satisfied. The iteration condition may be a convergence criterion or a predetermined number of iterations, as examples. In some cases, the generator sends the same fake image to all discriminators. In some cases, the generator sends the same fake image to at least two discriminators. In some cases, the generator sends a different fake image to each discriminator. In some cases, the generator can send a same image to multiple discriminators during one iteration and then send each discriminator its own image during a subsequent iteration.

The above-described method may be carried out for other data types, such as documents, genomic data, and medical records, instead of or in addition to images. In addition, each model at the generator neural network 210 can be updated in operation 340 and used to generate a corresponding fake image (or other data type) in operation 310 (and iterations). For example, the method can include generating, at the generator neural network 210, a fake image using each model; sending appropriate one or ones of the fake images to at least one discriminator node; receiving at least one gradient generated with respect to the fake images; and updating the models at the generator neural network using the received at least one gradient.

The fake images generated using the different models can all be sent to each discriminator node or only certain one or ones may be sent, depending on the preferred modality at the discriminator node (e.g., the modality to which the discriminator has a corresponding training data set for). The preferred modality (or modalities) may be indicated in a registration step as described above. In some cases, all models are updated upon receiving the at least one gradient. In some cases, a select subset of the models is updated when the at least one gradient is received.

Returning to FIG. 2, the central computing system 200 can further include a data access system 240, which can include a synthetic record resource 250 and a data access module 260, which can include a processor and storage with instructions supporting third party access to synthetic data generated by the generator neural network 210. Application programming interfaces (APIs) can be provided as part of the data access system.

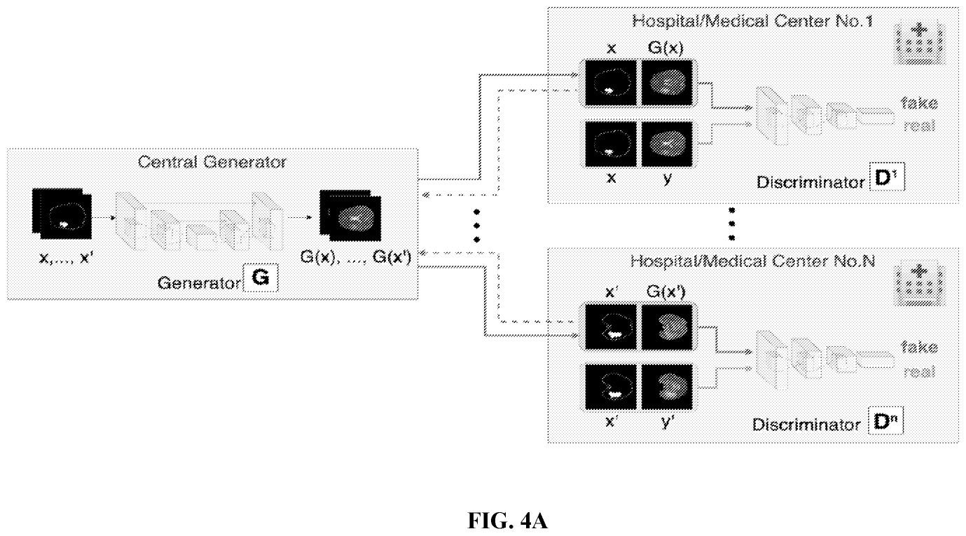

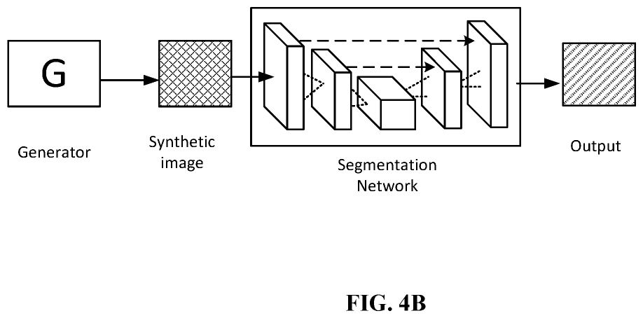

FIGS. 4A and 4B illustrate an overview of an AsynDGAN architecture applied to privacy-restricted image applications. As illustrated in FIG. 4A, a generator learns a joint distribution from different data sets that belong to different medical entities. Then, as illustrated in FIG. 4B, the generator can be used as an image provider to train a specific task, because the synthetic images are expected to share the same or similar distribution as the real images.

Referring to FIG. 4A, the centralized generator, denoted as G, takes task-specific inputs (segmentation masks in the experiments described herein) and generates synthetic images to fool the discriminators. The local discriminators, denoted as D¹ to Dⁿ, learn to differentiate between the local real images and the synthetic images from G. Due to the sensitivity of patients' images, the real images in each medical center may not be accessed from outside. The AsynDGAN architecture is naturally capable of avoiding such limitation because only the specific discriminator in the same medical entity needs to access the real images. In this way, the real images in local medical entities will be kept privately. Only synthetic images, losses, masks, and gradients are transferred between the centralized generator and the medical entities.

The objective of a classical conditional GAN is:

$${min}_{G} {max}_{D} V ( D , G ) = {𝔼}_{x \sim {s}_{j} ( x )} {𝔼}_{y \sim {p}_{data} ( y \vert x )} [ log D ( y \vert x ) ] + {𝔼}_{\hat{y} \sim {p}_{\hat{y}} ( \hat{y} \vert x )} [ log ( 1 - D ( \hat{y} \vert x ) ) ] ,$$

where D represents the discriminator and G is the generator. G aims to approximate the conditional distribution p_{data}(y|x) so that D cannot tell if the data is ‘fake’ or not. The hidden variable x is an auxiliary variable to control the mode of generated data. In reality, x is usually a class label or a mask that can provide information about the data to be generated. Instead of providing Gaussian noise z as an input to the generator, the noise is provided only in the form of dropout (the dropping of units in a neural network, e.g., randomly setting the outgoing edges of hidden neurons to 0), which is applied to several layers of the generator of the AsynDGAN at both training and test time.

As explained with respect to FIGS. 1A and 1B, a GAN estimates generative distribution via an adversarial supervisor, the discriminator. To obtain a task-specified distribution, an auxiliary variable x is fed to G together with noise prior z to serve as condition variable. Thus, the learning task reduces to a conditional distribution estimation. Although a conditional distribution estimation is described in detail herein (e.g., due to the nature of health entities learning problems), the described AsynDGAN framework can be easily adopted into general GAN learning tasks.

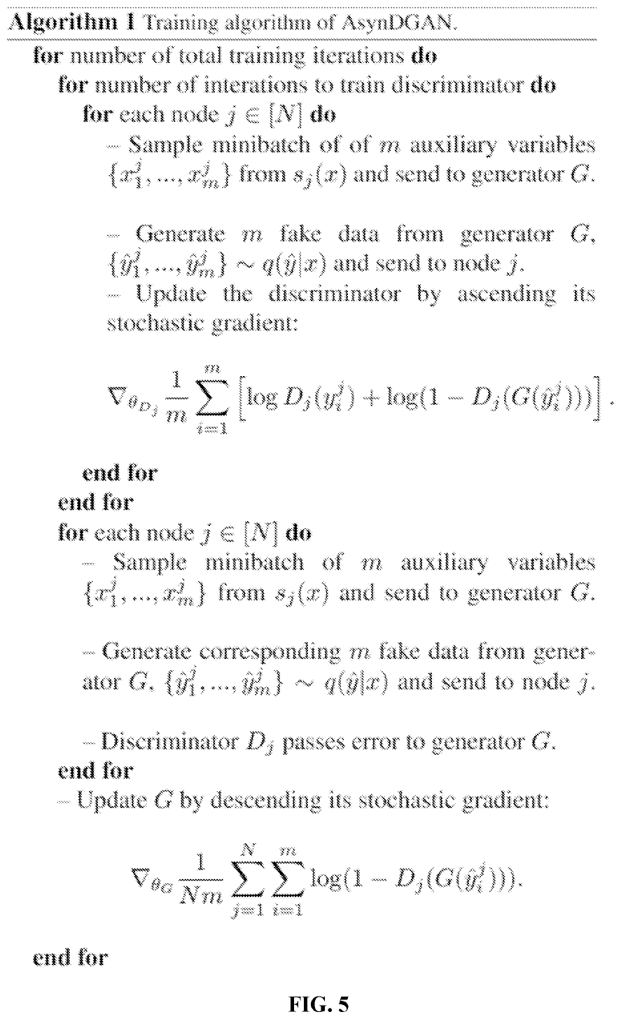

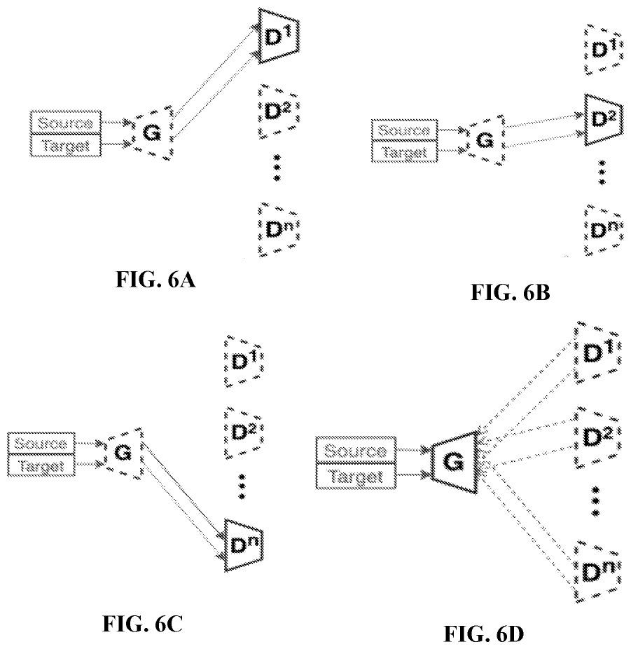

FIG. 5 shows an example training algorithm for a distributed GAN; and FIGS. 6A-6D illustrate an optimization process for a distributed GAN. Referring to FIGS. 5 and 6A-6D, a distributed GAN (“AsynDGAN”) can be performed where, in each iteration, a randomly sampled tuple (x, y) is provided to the system. Here, x denotes the input label which observed by the generator, and y is the real image only accessible by medical entities.

In the AsynDGAN framework, the generator is supervised by N different discriminators. Each discriminator is associated with a subset of data sets. Such a setting is quantified using a mixture distribution on auxiliary variable x. Here, instead of given a naive s(x), the distribution of x becomes

$$s ( x ) = {\sum }_{j ∈ [ N ]} {π}_{j} {s}_{j} ( x )$$

For each sub-distribution, there is a corresponding discriminator Dⱼ which only receives data generated from prior sⱼ(x). Therefore, the loss function of the AsynDGAN becomes:

$${min}_{G} {max}_{D} V ( D , G ) = {\sum }_{j ∈ [ N ]} {π}_{j} \{ {𝔼}_{x \sim {s}_{j} ( x )} {𝔼}_{y \sim {p}_{data} ( y \vert x )} [ log D ( y \vert x ) ] + {𝔼}_{\hat{y} \sim {p}_{\hat{y}} ( \hat{y} \vert x )} [ log ( 1 - D ( \hat{y} \vert x ) ) ] \}$$

In FIGS. 6A-6B, the solid arrows show gradient flow during the backward pass of the iterative update procedure. The solid block indicates that it is the block being updated, while the dotted blocks mean that those blocks are frozen during that update step. Following the algorithm, the network blocks are updated iteratively in the following order:

First, as reflected in FIGS. 6A-6C, a D-update is performed by calculating the adversarial loss for j-th discriminator Dⱼ and updating Dⱼ, where j=1, 2, . . . , N. Next, as reflected in FIG. 6D, a G-update is performed. That is, after updating all discriminators, G will be updated using the adversarial loss Σⱼ₌₁^{N} loss(Dⱼ). In the figures, the source is the source mask and the target is the target real image.

A cross entropy loss is applied in the example algorithm; however, it should be understood that other techniques may be used, including but not limited to variants of GAN loss including Wasserstein distance and classical regression loss.

It should also be noted that the sequence for transmitting to the discriminators could be varied. For example, the D-update can be transmitted to update the discriminators one by one or all the discriminators can be updated all at once, or some subset is updated at once and then another one or more are updated subsequent to the update to the subset. In addition, the feedback by each discriminator can be received one by one, all at once, or some other grouping.

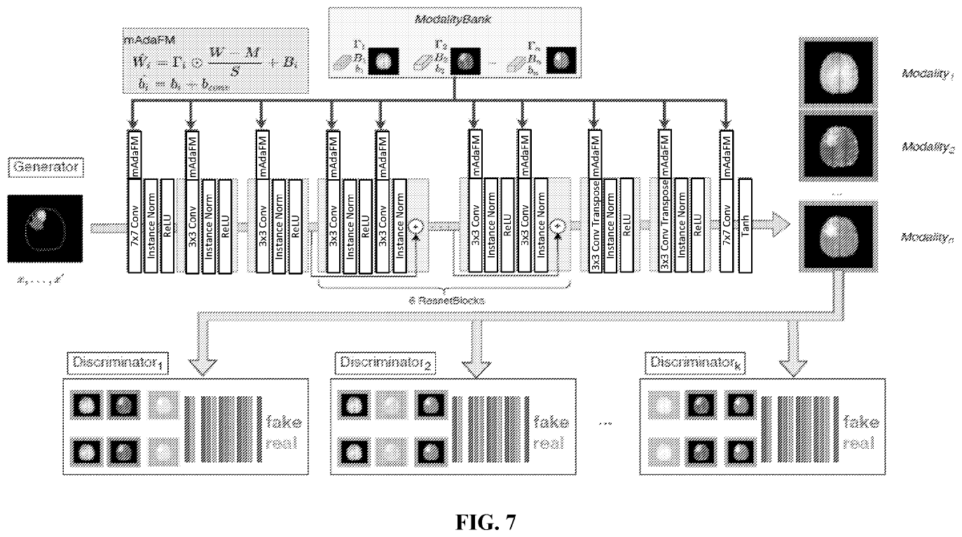

FIG. 7 illustrates an AsynDGAN architecture incorporating a modality bank. The described AsynDGAN with modality bank can be used to adaptively generate multi-modality images for various downstream tasks (such as segmentation) by incorporating a domain-specific modulation parameters bank.

When the central generator is used for multi-modality data, the registration process described above with respect to FIG. 2 can further include indicating which imaging modalities the site prefers for the fake data and may include a request to add a new modality to the modality bank, which would cause the central computing system to add a new parameter set to the parameter bank of the modality bank.

For this architecture, the generation of multi-modality data can be formulated as a style modulation task. A modified adaptive filter modulation (mAdaFM) is used to modulate the statistics of the weight in convolutional kernels to synthesis multi-modality images, even with severe style difference.

Here, the reparameterizations of domain-specific modulation parameters bank is given as Φ={Γ₁, B₁, b₁, r₂, B₂, b₂, . . . , Γₙ, Bₙ, bₙ,}, where n represents the types of modality images. The original convolution layers:

y=f_{conv}(x;W,b_{conv})

could be present as:

$${y}_{i} = {f}_{c o n v} ( x ; {\hat{W}}_{i} , {\hat{b}}_{i} ) , i ∈ [ 1 , n ]$$

$${\hat{W}}_{i} = {Γ}_{i} ⊙ \frac{W - M}{S} + {B}_{i} ,$$

$${\hat{b}}_{i} = {b}_{i} + {b}_{c o n v}$$

where M,S∈ [symbol] ^{C}^{ₒᵤₜ}^{×C}^{ᵢₙ} denote the mean and standard deviation of the weight in the convolutional kernel, respectively. The Γ₁, B∈ [symbol] ^{C}^{ₒᵤₜ}^{×C}^{ᵢₙ} and b_{conv}∈ [symbol] ^{C}^{ₒᵤₜ} represent learnable modality-specific parameters.

The generator can be first pre-trained on one initial modality and then the W and b of each convolutional/deconvolutional layer are fixed. The learnable style parameters (γ, β, b_{conv}) in mAdaFM, represented here by Γᵢ, Bᵢ, bᵢ, are used to modulate the Conv/Deconv layers. Therefore, it is possible to just store one generator along with a small set of style parameters for the synthesis of multi-modality images. In particular, as described above, the learnable parameters Γᵢ, Bᵢ, bᵢ are style parameters assigned to each modality i, and are trained to transform the fixed convolutional kernel to Ŵᵢ, \hat{b}ᵢ to synthesis images for the target modality.

In the example implementation of an AsynDGAN with modality bank, the illustrated modalities include MR imaging with several acquisition parameters, non-contrast/contrast CT, Ultrasound, and PET (positron emission tomography). These modalities can help to extract features from different perspectives and provide comprehensive information in medical image analysis.

As illustrated in the implementation of FIG. 7, one central generator G, denoted as Generator, takes task-specific inputs (e.g. segmentation masks in the example use case) and generates synthetic images to fool the discriminators. The multiple distributed discriminators (D₁, D₂, . . . , Dₖ) can be located in different medical entities, where k denotes the number of medical data centers that are involved in the learning framework. The central generator includes a modality bank with sets of parameters (Γi,Bi,bi for modality i) and uses these sets of parameters to output multi-modality synthetic images by different adaptive parameters. Each discriminator learns to differentiate between the real images of current medical entity and synthetic images from G. More than one modality may be used by each discriminator (e.g., where the medical entity has or uses different modalities).

The architecture ensures that Discriminator k (Dₖ) deployed in the k^{th} medical entity only has the access to its local dataset, while not sharing any real image data outside the entity. During the learning process, only synthetic images, masks, and losses are transferred between the central generator and the discriminators. Such design naturally complies with privacy regularization and keeps the patients' sensitive data safe. After training, the generator can be used as an image provider to generate training samples for some down-stream tasks. Assuming the distribution of synthetic images is the same or similar to that of the real images, it is possible to generate one unified large dataset which approximately equals to the union of all the datasets in medical entities. In this way, all private image data from each entity are utilized without sharing.

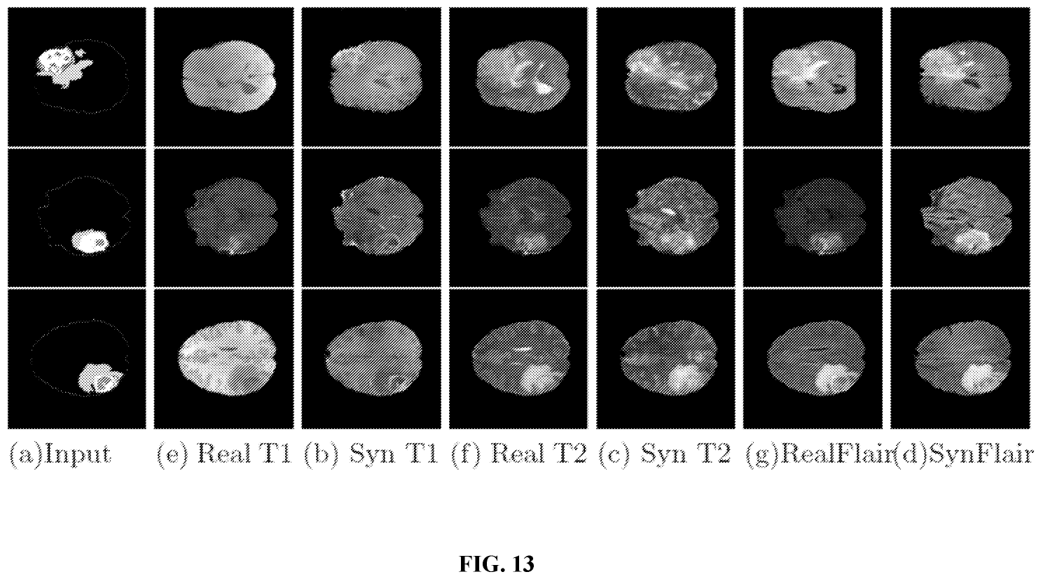

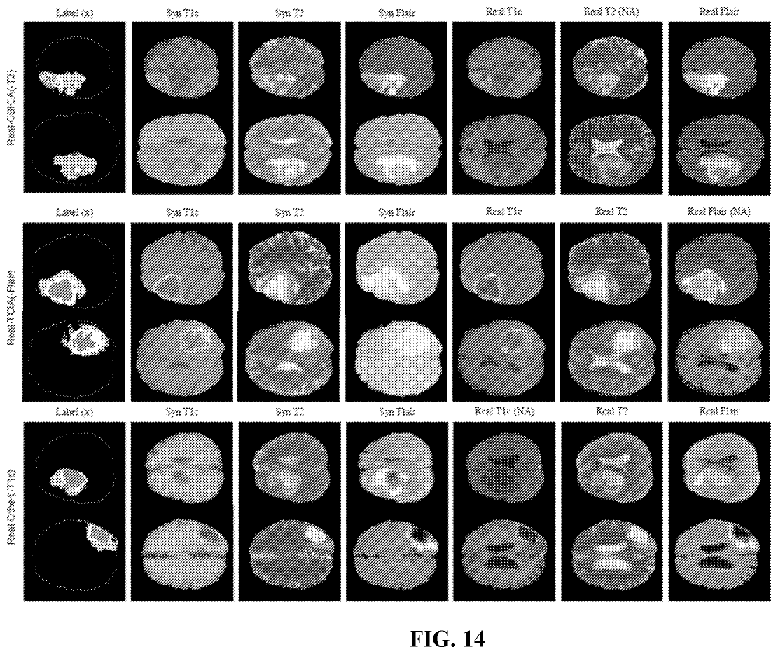

FIGS. 13 and 14 represent the evaluation experiments for the multi-modality implementation for generated samples in segmentation tasks.

Theoretical Analysis

Lemma 1. When generator G is fixed, the optimal discriminator Dⱼ(y|x) is:

$${D}_{j} ( y \vert x ) = \frac{p ( y \vert x )}{p ( y \vert x ) + q ( y \vert x )}$$

Suppose in each training step the discriminator achieves its maxima criterion in Lemma 1, the loss function for the generator becomes:

$${min}_{G} V ( G ) = {𝔼}_{y} {𝔼}_{x \sim {p}_{data} ( y \vert x )} [ log D ( y \vert x ) ] + {𝔼}_{\hat{y} \sim {p}_{\hat{y}} ( \hat{y} \vert x )} [ log ( 1 - D ( \hat{y} \vert x ) ) ] = {\sum }_{j ∈ [ N ]} {π}_{j} {\int }_{y} {s}_{j} ( x ) {\int }_{x} p ( y \vert x ) log \frac{p ( y \vert x )}{p ( y \vert x ) + q ( y \vert x )} + q ( y \vert x ) log \frac{q ( y \vert x )}{p ( y \vert x ) + q ( y \vert x )} d x d y$$

Assuming in each step, the discriminator always performs optimally, it can be seen that the generative distribution G seeks to minimize the loss by approximating the underlying distribution of data.

Theorem 1. Suppose the discriminators D_{1˜N} always behave optimally (denoted as D_{1˜N}*), the loss function of generator is global optimal iff q(y,x)=p(y,x) where the optimal value of V(G, D_{1˜N}*), is −log 4.

Remark 1. In distributed learning setting, data from different nodes are often dissimilar. Consider the case where Ω(sⱼ(x))∩Ω(sₖ(y))=Ø, for k≠j, the information for p(y|x), y∈Ω(sⱼ(x)) will be missing if the jth node is lost. The behavior of trained generative model is unpredictable when receiving auxiliary variables from unobserved distribution sⱼ(x). The AsynDGAN framework provides a solution for unifying different data sets by collaborating multiple discriminators.

Experimental Results and Comparison Tests

In the experiments, the AsynDGAN framework is applied to segmentation tasks to illustrate its effectiveness. The U-Net, as described by Olaf Ronneberger et al. (“Unet: Convolutional networks for biomedical image segmentation,” In International Conference on Medical image computing and computer-assisted intervention, pages 234-241, Springer 2015), is used as the segmentation model, and details about G and Ds designed for segmentation tasks are described below.

For segmentation tasks, the centralized generator is an encoder-decoder network that consists of two stride-2 convolutions (for downsampling), nine residual blocks, and two transposed convolutions. All non-residual convolutional layers are followed by batch normalization and the ReLU activation. All convolutional layers use 3×3 kernels except the first and last layers that use 7×7 kernels.

In the AsynDGAN framework, the discriminators are distributed over N nodes (e.g., hospitals, mobile devices). Each discriminator Dⱼ only has access to data stored in the j^{th} node thus discriminators are trained in an asynchronized fashion. Each discriminator has the same structure as that in PatchGAN (see Phillip Isola et al., “Image-to-Image Translation with Conditional Adversarial Networks,” 2016). The discriminator individually quantifies the fake or real value of different small patches in the image. Such architecture assumes patch-wise independence of pixels in a Markov random field fashion, and can capture the difference in geometrical structures such as background and tumors.

The experiments first involve a synthetic data set to show how the AsynDGAN learns a mixed distribution from different subsets.

The synthetic data set is generated by mixing 3 one-dimensional Gaussian. In particular, a set x∈{1,2,3} is generated with equal probabilities. Given x, the random variable y is generated from y=y₁1ₓ₌₁+y₂1ₓ₌₂+y₃1ₓ₌₃ where 1ₑᵥₑₙₜ is the indicator function and y₁˜ [symbol] (−3,2), y₂˜ [symbol] (1,1), y₃˜ [symbol] (3,0.5). Suppose the generator learns the conditional distribution of y:p(y|x) perfectly, the histogram should behave similarly to the shape of the histogram of mixture gaussian.

In the synthetic learning phase, the 9-Blocks ResNet architecture for the generator and multiple discriminators having the same structure as that in Patch-GAN with patch size 70×70 were used (see Kaiming He et al. “Deep residual learning for image recognition,” In Proceedings of the IEEE conference on computer vision and pattern recognition, pages 770-778, 2016; and Phillip Isola et al., “Image-to-Image Translation with Conditional Adversarial Networks,” 2016). A resize and crop strategy was applied to resize the input slices as 286×286 and then randomly crop the image to 256×256. In addition to the GAN loss and the L1 loss, perceptual loss, as described by Justin Johnson et al., in “Perceptual losses for real-time style transfer and super-resolution,” In European Conference on Computer Vision, 2016, was used.

Minibatch SGD was used and the Adam solver (optimizer) applied with a learning rate of 0.0002 and momentum parameters β1=0.5, β2=0.999 (see Diederik P Kingma and Jimmy Ba, “Adam: A method for stochastic optimization,” arXiv preprint arXiv:1412.6980, 2014).

The batch size used in AsynDGAN depends on number of discriminators. A batch size of 3 was used for the BraTS2018 data set and a batch size of 1 was used for the Multi-Organ data set.

In the segmentation phase, images of 224×224 were randomly cropped with a batch size of 16 as input. The model is trained with the Adam optimizer using a learning rate of 0.001 for 50 epochs in brain tumor segmentation and 100 epochs in nuclei segmentation. To improve performance, data augmentation was used in all experiments, including random horizontal flip and rotation in tumor segmentation and additional random scale and affine transformation in nuclei segmentation.

Through an experiment on the synthetic data set, it can be seen that the described synthetic learning framework can learn a mixture of Gaussian distribution from different subsets. The quality of learning distribution is compared in 3 settings: (1) Syn-All. Training a regular GAN using all samples in the data set. (2) Syn-Subset-n. Training a regular GAN using only samples in local subset n, where n∈{1,2,3}. 3) AsynDGAN. Training the AsynDGAN using samples in a distributed fashion.

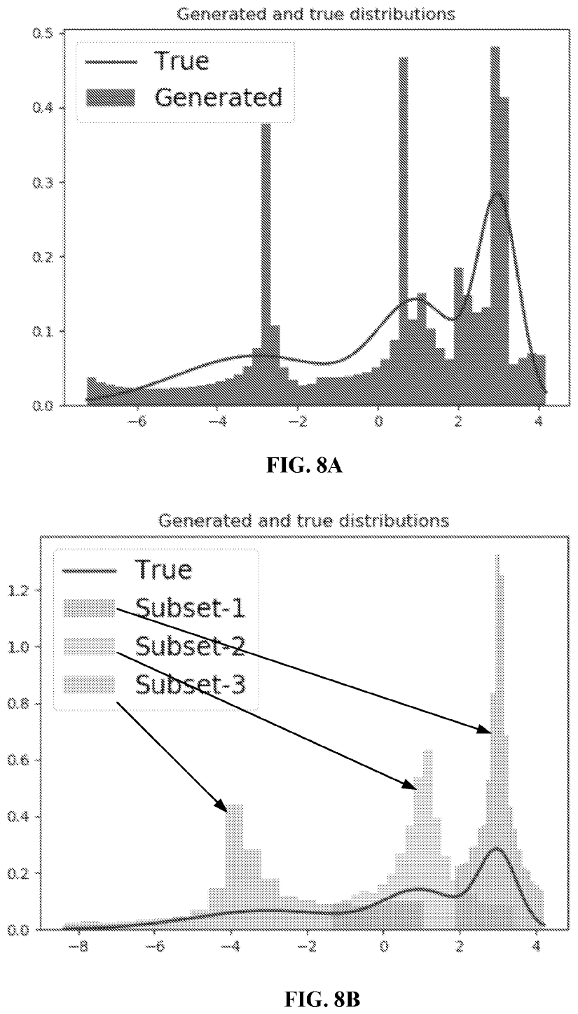

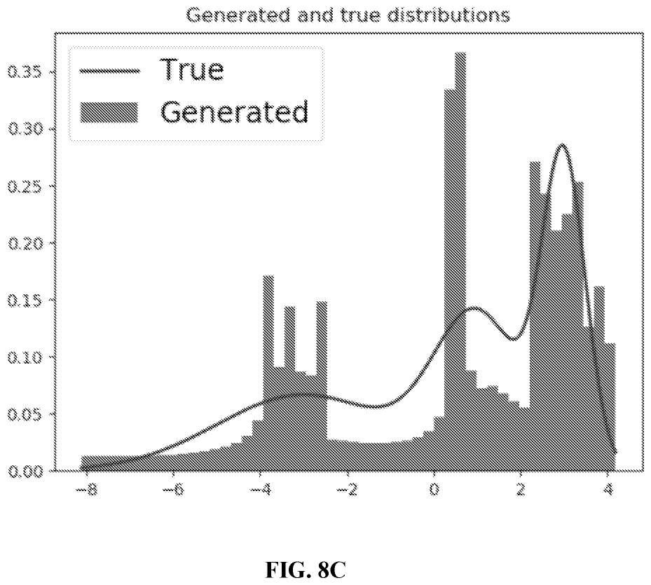

FIGS. 8A-8C show plots of the learned distributions in accordance with an experiment on synthetic data, where FIG. 8A shows a plot of the Syn-All setting; FIG. 8B shows a plot of the Syn-Subset-n setting; and FIG. 8C shows a plot of the AsynDGAN setting. As can be seen with reference to FIG. 8B, any local learning fits just one mode Gaussian due to the restriction of local information. In contrast, with reference to FIGS. 8A and 8C, it can be seen that AsynDGAN is able to capture global information and thus has a comparable performance with the regular GAN using the union of separated data sets (Syn-All).

Testing of brain tumor segmentation was performed on BraTS2018 data set.

The BraTS2018 data set comes from the Multimodal Brain Tumor Segmentation Challenge 2018 and contains multi-parametric magnetic resonance imaging mpMRI) scans of low-grade glioma (LGG) and high-grade glioma (HGG) patients. There are 210 HGG and 75 LGG cases in the training data, and each case has four types of MRI scans and three types of tumor subregion labels. In the experiments, 2D segmentation was performed on T2 images of the HGG cases to extract the whole tumor regions. The 2D slices with tumor areas smaller than 10 pixels are excluded for both GAN training and segmentation phases. In the GAN synthesis phase, all three labels are utilized to generate fake images. For segmentation, the focus is on the whole tumor (regions with any of the three labels).

As mentioned above, there are 210 HGG cases in the training data. Because there was no access to the test data of the BraTS2018 Challenge, the 210 HGG cases in the training data were split into train (170 cases) and test (40 cases) sets. The train set was then sorted according to the tumor size and divided into 10 subsets equally, which was treated as data in 10 distributed medical entities. There are 11,057 images in the training set and 2,616 images in the test set.

The same metrics as described in the BraTS2018 Challenge are used to evaluate the segmentation performance of brain tumor: Dice score (Dice), sensitivity (Sens), specificity (Spec), and 95% quantile of Hausdorff distance (HD95). The Dice score, sensitivity (true positive rate) and specificity (true negative rate) measure the overlap between ground-truth mask G and segmented result S. They are defined as

$$Dice ( G , S ) = \frac{2 \vert G ⋂ S \vert}{\vert G \vert + \vert T \vert}$$

$$Sens ( G , S ) = \frac{\vert G ⋂ S \vert}{\vert G \vert}$$

$$Spec ( G , S ) = \frac{\vert ( 1 - G ) ⋂ ( 1 - S ) \vert}{\vert 1 - G \vert}$$

The Hausdorff distance evaluates the distance between boundaries of ground-truth and segmented masks:

$$H D ( G , S ) = max \{ \begin{matrix}sup \\ x ∈ \partial G\end{matrix} \begin{matrix}inf \\ y ∈ \partial S\end{matrix} d ( x , y ) , \begin{matrix}sup \\ y ∈ \partial S\end{matrix} \begin{matrix}inf \\ x ∈ \partial G\end{matrix} d ( x , y ) \}$$

where ∂ means the boundary operation, and d is Euclidean distance. Because the Hausdorff distance is sensitive to small outlying subregions, the 95% quantile of the distances are used instead of the maximum. For ease of comparison, a 2D segmentation task instead of a 3D segmentation task is used for the BraTS2018 Challenge. These metrics were computed on each 2D slices and an average on all 2D slices were taken in the test set.

The following segmentation experiments were conducted:

Real-All. Training using real images from the whole train set (170 cases).

Real-Subset-n. Training using real images from the n-th subset (medical entity), where n=1, 2, . . . , 10. There are 10 different experiments in this category.

Syn-All. Training using synthetic images generated from a regular GAN. The GAN is trained directly using all real images from the 170 cases.

AsynDGAN. Training using synthetic images from the AsynDGAN. The AsynDGAN is trained using images from the 10 subsets (medical entities) in a distributed fashion.

In all experiments, the test set remains the same for fair comparison. It should be noted that in the Syn-All and AsynDGAN experiments, the numbers of synthetic images are the same as that of real images in Real-All. The regular GAN has the same generator and discriminator structures as AsynDGAN, as well as the hyper-parameters. The only difference is that AsynDGAN has 10 different discriminators, and each of them is located in a medical entity and only has access to the real images in one subset.

The quantitative brain tumor segmentation results are shown in Table 2.

TABLE 2
Brain tumor segmentation results.

| Method | Dice ↑ | Sens ↑ | Spec ↑ | HD95 ↓ |
|---|---|---|---|---|
| Real-All | 0.7485 | 0.7983 | 0.9955 | 12.85 |
| Real-Subset-1 | 0.5647 | 0.5766 | 0.9945 | 26.90 |
| Real-Subset-2 | 0.6158 | 0.6333 | 0.9941 | 21.87 |
| Real-Subset-3 | 0.6660 | 0.7008 | 0.9950 | 21.90 |
| Real-Subset-4 | 0.6539 | 0.6600 | 0.9962 | 21.07 |
| Real-Subset-5 | 0.6352 | 0.6437 | 0.9956 | 19.27 |
| Real-Subset-6 | 0.6844 | 0.7249 | 0.9935 | 21.10 |
| Real-Subset-7 | 0.6463 | 0.6252 | 0.9972 | 15.60 |
| Real-Subset-8 | 0.6661 | 0.6876 | 0.9957 | 18.16 |
| Real-Subset-9 | 0.6844 | 0.7088 | 0.9953 | 18.56 |
| Real-Subset-10 | 0.6507 | 0.6596 | 0.9957 | 17.33 |
| Syn-All | 0.7114 | 0.7099 | 0.9969 | 16.22 |
| AsynDGAN | 0.7043 | 0.7295 | 0.9957 | 14.94 |

The model trained using all real images (Real-All) is the ideal case representing access to all data. It is the baseline and achieves the best performance. As can be seen in Table 2, compared with the ideal baseline, the performance of models trained using data in each medical entity (Real-Subset-1˜10) degrades significantly because the information in each subset is limited and the number of training images is much smaller. The AsynDGAN can learn from the information of all data during training, although the generator does not “see” the real images. And it is possible to generate as many synthetic images as desired to train the segmentation model. Therefore, the model (AsynDGAN) outperforms all models using single subset. For reference, the results using synthetic images from regular GAN (Syn-All) is also shown, which is trained directly using all real images. The AsynDGAN has the same performance as the regular GAN, but has no privacy issue because the AsynDGAN does not collect real image data from medical entities.

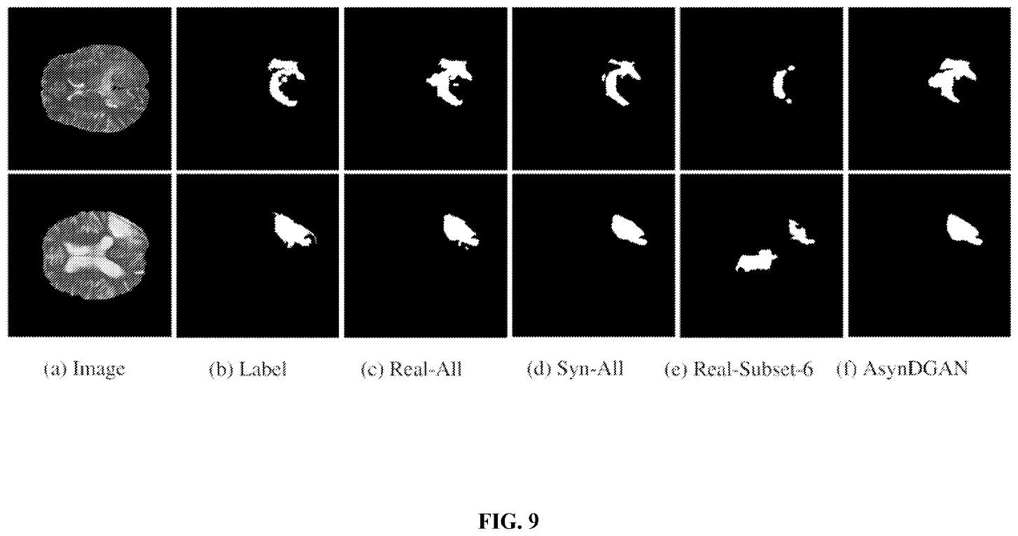

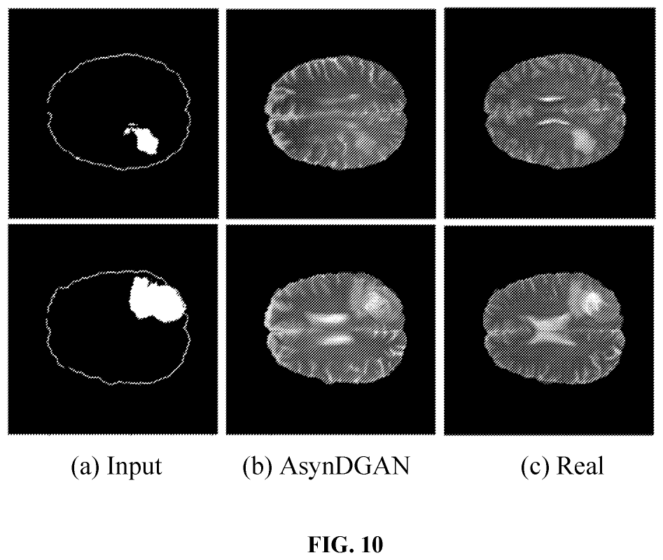

FIG. 9 shows images from the quantitative brain tumor segmentation results with respect to each of the four methods of the experiment. Referring to FIG. 9, panel A shows two test images; panel B shows ground-truth labels of the tumor region for the corresponding two test images; panel C shows results of a model trained on all real images; panel D shows results of a model trained on synthetic images from regular GAN; panel E shows results of a model trained on real images from subset-6; and panel F shows results of a model trained on synthetic images from the AsynDGAN.

FIG. 10 shows examples of synthetic images from AsynDGAN as part of the quantitative brain tumor segmentation experiment. Referring to FIG. 10, panel A shows the input of the AsynDGAN network; panel B shows the synthetic images of AsynDGAN based on the input; and panel C shows real images for comparison.

Testing of multiple organ nuclei segmentation was also performed. This testing was performed on a Multi-Organ data set proposed by Kumar et al. (“A dataset and a technique for generalized nuclear segmentation for computational pathology,” IEEE transactions on medical imaging, 36(7):1550-1560, 2017). for nuclei segmentation. There are 30 histopathology images of size 1000×1000 from 7 different organs. The train set contains 16 images of breast, liver, kidney, and prostate (4 images per organ). The same organ test set contains 8 images of the above four organs (2 images per organ) while the different organ test set has 6 images from bladder, colon, and stomach. In the current experiments, the focus is on the four organs that exist both in the train and test sets, and color normalization was performed for all images. Two training images of each organ is treated as a subset that belongs to a medical entity.

Here, it is assumed that the training images belong to four different medical entities and each entity has two images of one organ.

For nuclei segmentation, a Dice score and the Aggregated Jaccard Index (AJI) were utilized:

$$A J I = \frac{{Σ}_{i = 1}^{n 𝒢} \vert {G}_{i} ⋂ S ( {G}_{i} ) \vert}{{Σ}_{i = 1}^{n 𝒢} \vert {G}_{i} ⋃ S ( {G}_{i} ) \vert + {Σ}_{k ∈ K} \vert {S}_{k} \vert}$$

where S(Gᵢ) is the segmented object that has maximum overlap with G, with regard to Jaccard index, K is the set containing segmentation objects that have not been assigned to any ground-truth object.

Similar to the testing of brain tumor segmentation, the following segmentation experiments were conducted:

Real-All. Training using the 8 real images of the train set.

Real-Subset-n. Training using 2 real images from each subset (medical entity), where n∈{breast, liver, kidney, prostate}.

Syn-All. Training using synthetic images from regular GAN, which is trained using all 8 real images.

AsynDGAN. Training using synthetic images from the AsynDGAN, which is trained using images from the 4 subsets distributively.

In all experiments, the same organ test set was used for evaluation.

The quantitative nuclei segmentation results are presented in Table 3.

TABLE 3
Nuclei segmentation results.

| Method | Dice ↑ | AJI ↑ |
|---|---|---|
| Real-All | 0.7833 | 0.5608 |
| Real-Subset-breast | 0.7340 | 0.4942 |
| Real-Subset-liver | 0.7639 | 0.5191 |
| Real-Subset-kidney | 0.7416 | 0.4848 |
| Resl-Subset-prostate | 0.7704 | 0.5370 |
| Syn-All | 0.7856 | 0.5561 |
| AsynDGAN | 0.7930 | 0.5608 |

As can be seen in Table 3, compared with models using single organ data, the AsynDGAN method achieves the best performance. The reason is that local models cannot learn the nuclear features of other organs. Compared with the model using all real images, the AsynDGAN has the same performance, which shows the effectiveness of the described method in this type of task. The result using regular GAN (Syn-All) is slightly worse than the AsynDGAN, probably because one discriminator is not good enough to capture different distributions of nuclear features in multiple organs. In AsynDGAN, each discriminator is responsible for one type of nuclei, which may be better for the generator to learn the overall distribution.

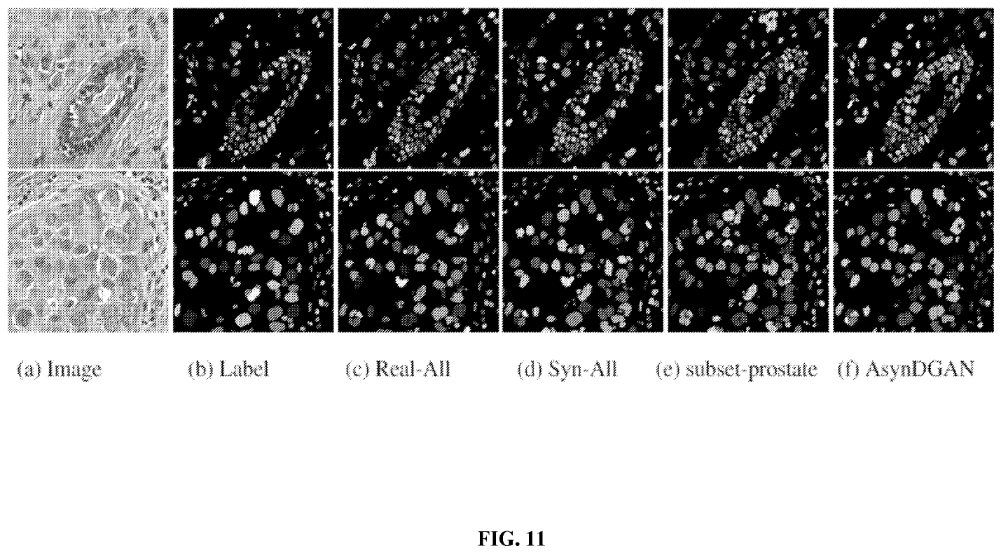

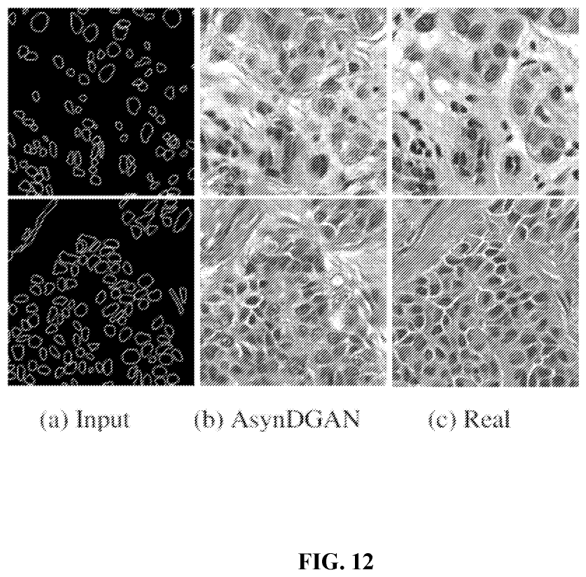

Several examples of synthetic images from AsynDGAN are shown in FIG. 12, and typical qualitative segmentation results are shown in FIG. 11.

FIG. 11 shows images from the nuclei segmentation results with respect to each of the four methods of the experiment. Referring to FIG. 11, panel A shows two test images; panel B shows ground-truth labels of nuclei for the corresponding two test images; panel C shows results of a model trained on all real images; panel D shows results of a model trained on synthetic images from regular GAN; panel E shows results of a model trained on real images of prostate; and panel F shows results of a model trained on synthetic images from the AsynDGAN.

FIG. 12 shows examples of synthetic nuclei images from AsynDGAN. Referring to FIG. 12, panel A shows the input of the AsynDGAN network; panel B shows the synthetic images of AsynDGAN based on the input; and panel C shows real images for comparison.

As reflected by the experimental results, it can be seen that the described distributed asynchronous discriminator GAN approach can learn a real image's distribution from multiple data sets without sharing the patient's raw data; is more efficient and requires lower bandwidth than other distributed deep learning methods; and can achieve higher performance compared to a segmentation model trained by one real data set, and almost the same performance compared to the segmentation model trained by all real data sets.

Experiments were also conducted on the AsynDGAN with modality bank configured such as described with respect to FIG. 7. The modality bank configured AsynDGAN was applied on the BraTS2018 data set. For a first evaluation, three heterogeneous data centers were modeled with three different modalities using different settings among the data centers and modalities. For a second evaluation, the ability and adaptability of a modality bank to complete missing modality across data centers was explored. The network was pretrained based on the images from the modality that a particular experiment did not use. For example, a pre-trained model learned from T1c modality images was used for the first experiment, while T1 modality images were used for the second experiment. Without loss of generality, image segmentation was adopted as the down-stream task for the synthetic images.

As mentioned above with respect to the experiments on the AsynDGAN, the BraTS2018 dataset comes from the Multimodal Brain Tumor Segmentation Challenge 2018. All images were acquired from the three different sources: (1) The Center for Biomedical Image Computing and Analytics (CBICA) (2) The Cancer Imaging Archive (TCIA) data center (3) Data from other sites (Other). Each case has four types of MRI scan modalities (T1, T1c, T2 and FLAIR) and three types of tumor sub-region labels. All modalities were aligned to a common space and resampled to 1 mm isotropic resolution. The 210 HGG cases in the challenge training set were split into train (170 cases) and test (40 cases) sets.

In the modality bank experiments, the method was evaluated to learn the distribution of all HGG cases across different data centers. In the GAN synthesis phase, all three labels were utilized to generate fake images. For segmentation, the focus was on the whole tumor region (union of all three labels). The image dataset used in each experiment share one or multiple modalities. Without loss of generality, T1+T2+FLAIR and T1c+T2+FLAIR modalities were respectively selected for the two experiments.

In the first experiment, the brain tumor segmentation results on the test set are shown in Table 4. Table 4 shows brain tumor segmentation results over the three heterogeneous and multi-modal (T1+T2+Flair) subsets.

| Method | Dice(%) ↑ | Sens(%) ↑ | Spec(%)↑ | HD95↓ |
|---|---|---|---|---|
| Real-All | 87.9 ± 8.5 | 85.6 ± 13.5 | 99.8 ± 0.3 | 10.51 ± 5.93 |
| FedML-All | 87.3 ± 8.4 | 85.22 ± 14.9 | 99.8 ± 0.2 | 12.6 ± 0.2 |
| Real-CBICA | 78.9 ± 19.6 | 75.7 ± 23.1 | 99.7 ± 0.2 | 16.45 ± 9.89 |
| Real-TCIA | 77.2 ± 12.1 | 82.1 ± 16.1 | 99.3 ± 0.4 | 12.68 ± 4.95 |
| Real-Other | 80.4 ± 12.9 | 80.7 ± 19.4 | 99.5 ± 0.3 | 22.33 ± 14.0 |
| AsynDGAN | 82.0 ± 17.6 | 81.9 ± 22.0 | 99.5 ± 0.6 | 13.93 ± 10.0 |
| ModalityBank | 84.4 ± 14.9 | 81.2 ± 17.9 | 99.8 ± 0.2 | 13.95 ± 13.07 |

It can be seen that the AsynDGAN with modality bank (“Modality Bank”) can learn the distributions and generate realistic multi-modality medical images across heterogeneous data centers. Specifically, the generator can generate realistic three channels (T1, T2, Flair) multi-modality images by learning from three heterogeneous data sources. The training data was split into 3 subsets based on the different sources of the data: (1) Real-CBICA, 88 cases collected from CBICA. (2) Real-TCIA, 102 cases collected from TCIA. (3) Real-Other, 20 cases collected not from CBICA nor TCIA. The model trained using all real images (Real-All) is the ideal case scenario represented access to all data. It is the baseline and achieves the best performance. Compared with the ideal baseline, the performance of the models trained only using data in each medical entity (Real-CBICA, Real-TCIA, Real-Other) degrades considerably. The FedML.ai library was used for FedML-All experiment to train the segmentation model. It can make use of real images from all three subsets thus its performance is slightly lower than Real-All.

Results using AsynDGAN with and without the modality bank are shown (“AsynDGAN” and “ModalityBank”, respectively). By using a modality bank, the domain-specific modulation parameters can better handle different modalities. The method can learn the information of all subsets during training, although the generator does not “see” the real images. Therefore, the modality bank outperforms all models learn using a single subset. Some examples of synthetic images from the method and corresponding real images are shown in FIG. 13. Worth noticing is that the number of one modality configuration parameters is 2.5M while the number of all frozen source parameters is 21M. With smaller trainable parameters, it is possible to learn and store the modalities configuration more efficiently.

FIG. 13 shows examples of synthetic images from AsynDGAN with modality bank as part of a multi-modality brain tumor segmentation experiment. Referring to FIG. 13, panel (a) shows examples of input to the central computing system, panels (b)-(d) show resulting synthetic multi-modality images output by the central computing system, and panels (e)-(g) show real multi-modality images of the corresponding modalities.

The second experiment shows that it is possible to learn the misaligned modality distribution and generate the complete multi-modality images. Specifically, the generator with modality bank could generate realistic three channel(T1c, T2, Flair) multi-modality images while the real datasets do not provide one of the three modalities.

For the experiment, the training data was split into 3 subsets based on the different sources of the data as described above where one of the modalities is skipped as follows: (1) Real-CBICA(n/a:T2) skip T2 modality. (2) Real-TCIA(n/a:Flair) skip Flair modality (3) Real-Other(n/a:T1c) skip T1c modality.

The brain tumor segmentation results about modality completion are shown in Table 5. Table 5: Brain tumor segmentation results over three datasets with missing modality (Tic/T2/Flair)

| Method | Dice(%) ↑ | Sens(%) ↑ | Spec(%) ↑ | HD95 ↓ |
|---|---|---|---|---|
| Real-CBICA(n/a: T2) | 78.0 ± 23.4 | 74.5 ± 25.9 | 99.7 ± 0.2 | 15.47 ± 14.2 |
| Real-TCIA(n/a: Flair) | 76.7 ± 15.3 | 72.8 ± 20.8 | 99.5 ± 0.8 | 15.64 ± 8.75 |
| Real-Other(n/a: T1c) | 80.9 ± 14.1 | 79.3 ± 18.8 | 99.6 ± 0.2 | 16.74 ± 9.41 |
| FedML-All | 82.9 ± 8.7 | 90.2 ± 13.1 | 99.2 ± 0.7 | 21.88 ± 11.52 |
| ModalityBank | 85.8 ± 10.9 | 83.8 ± 16.6 | 99.7 ± 0.2 | 14.71 ± 7.99 |
| Completed-CBICA (syn: T2) | 83.0 ± 14.6 | 79.9 ± 19.0 | 99.7 ± 0.2 | 15.64 ± 9.93 |
| Completed-TCIA (syn: Flair) | 85.5 ± 10.4 | 83.3 ± 14.3 | 99.7 ± 0.1 | 15.02 ± 8.35 |
| Completed-Other (syn: T1c) | 80.9 ± 15.3 | 80.6 ± 19.1 | 99.6 ± 0.2 | 16.93 ± 11.8 |

As can be seen, segmentation performance dropped when the segmentation network only learns from the real data center with missing modality while completed multi-modality images generated by the AsynDGAN with modality bank (“ModalityBank”) could help the segmentation network to achieve much higher results. The method has the best performance compared with the real images from one data center and the federated learning method. Though FedML-All could learn the real distribution across all data centers, the architecture was not able to adapt to the discrepancy of missing modalities, therefore it has the worst performance.

By providing the missing modality images for each of the datasets, the completed dataset would also outperform the counterpart of the real dataset. From the results it appears that T2 and Flair may contribute more to the whole tumor segmentation task since learning from the smallest subset Real-Other(n/a:T1c) achieves higher performance compared with learning from the other subsets with missing T2 or Flair. As a result, there is no significant difference between Completed-Other (syn:T1c) and Real-Other(n/a:T1c) by introducing the synthetic T1c images.

FIG. 14 shows examples of synthetic images from AsynDGAN with modality bank as part of a multi-modality brain tumor segmentation experiment using missing modality data sets. In FIG. 14, the 3 sections are corresponding to three data centers, respectively. The column of the real image labeled as NA (not available) indicates the missing modality in that center during the training of the AsynDGAN with modality bank. The first observation is that the method can still learn to generate multiple modalities when centers have a missing modality. It also appears that the synthetic images may not have the same global context as the real images, for example, the generated brains may have different shapes of ventricles. This is due to the lack of information about other tissues outside the tumor region in the input of the G. On one hand, this variation is good for privacy preservation. On the other hand, for missing modality completion, the synthetic modality may have a different context from the real modalities. However, this limitation seems not critical to the segmentation task, since the results in Table 5 shows clear improvement after the missing modality completion.

Although the subject matter has been described in language specific to structural features and/or acts, it is to be understood that the subject matter defined in the appended claims is not necessarily limited to the specific features or acts described above. Rather, the specific features and acts described above are disclosed as examples of implementing the claims, and other equivalent features and acts are intended to be within the scope of the claims.

What is claimed is:

1. An asynchronous distributed generative adversarial network (AsynDGAN) comprising:
a central computing system comprising a generator neural network, an aggregator, and a network interface; and
at least two discriminator nodes, each discriminator node having its own corresponding training data set,
wherein the central computing system communicates with each of the at least two discriminator nodes via the network interface and aggregates data received from the at least two discriminator nodes, via the aggregator, to update a model for the generator neural network.

2. The AsynDGAN of claim 1, further comprising a modality bank storing style parameters for a plurality of models, including the model for the generator neural network, wherein the central computing system updates each of the plurality of models using a same data received from the at least two discriminator nodes.

3. The AsynDGAN of claim 2, wherein the style parameters for the plurality of models correspond to at least two respective image modalities.

4. The AsynDGAN of claim 1, wherein the central computing system further comprises a data access system providing access to synthetic data generated by the generator neural network.

5. A training method for the asynchronous distributed generative adversarial network of claim 1, the training method comprising:
generating, at a generator neural network, a fake image using a model;
sending the fake image to at least one discriminator node of a plurality of discriminator nodes, each discriminator node of the plurality of discriminator nodes having its own training data set;
receiving at least one gradient generated with respect to the fake image, each gradient being from a corresponding discriminator node of the at least one discriminator node of the plurality of discriminator nodes;
updating the model at the generator neural network using the received at least one gradient; and
iterating the generating of an updated fake image, sending the updated fake image to one or more of the discriminator nodes, and the updating of the model using new gradients received from the one or more of the discriminator nodes until an iteration condition is satisfied.

6. The method of claim 5, further comprising:
generating, at the generator neural network, at least a second fake image of a different modality than the fake image by using a corresponding model;
sending the second fake image to at least one discriminator node of the plurality of discriminator nodes; and
updating the corresponding model using a same received at least one gradient as used to update the model.

7. The method of claim 5, wherein updating the model at the generator neural network comprises updating style parameters for the model.

8. The method of claim 5, wherein updating the model at the generator neural network comprises adjusting weights for the generator neural network.

9. The method of claim 5, further comprising:
receiving, at the generator neural network a training data request from a discriminator node of the plurality of discriminator nodes; and
in response to receiving the training data request, generating a training synthetic data and transmitting the training synthetic data to the discriminator node.

10. The method of claim 5, wherein a same fake image is sent to each discriminator node of the plurality of discriminator nodes.

11. The method of claim 5, wherein a same fake image is sent to at least two discriminator nodes of the plurality of discriminator nodes.
